(19) United States
(12) Patent Application Publication
(10) Pub. No.: US 2026/0241129 A1
(43) Pub. Date: Aug. 20, 2026

(54) AUTOMATED CUFF PRESSURE MODULATION SYSTEM FOR ENDOTRACHEAL TUBES

(71) Applicant: CUFFWAY LLC, ELGIN, SC (US)

(72) Inventors: Shrihan Ganesh Babu, Elgin, SC (US); Stephan Nieuwoudt, Fairview Park, OH (US)

(21) Appl. No.: 19/544,888

(22) Filed: Feb. 19, 2026

Related U.S. Application Data
(60) Provisional application No. 63/760,577, filed on Feb. 19, 2025.

(51) Int. Cl. A61M 16/04; A61M 16/00; A61M 16/20

(52) U.S. Cl. CPC A61M 16/044; A61M 16/0003; A61M 16/026; A61M 16/202; A61M 16/209; A61M 2016/0018; A61M 2016/0027; A61M 2205/18; A61M 2205/3303; A61M 2205/3327; A61M 2205/3331; A61M 2205/502; A61M 2205/52; A61M 2210/1025; A61M 2230/005; A61M 2230/40

(57) ABSTRACT

Automated cuff pressure modulation systems are disclosed herein. In some embodiments, a cuff pressure modulation device for modulating cuff pressure of a cuff integrated with an airway tube includes a pressure sensor configured to track a real-time respiration pattern, a cuff pump configured to selectively inflate or deflate the cuff, a memory storing a minimum cuff pressure threshold and a maximum cuff pressure threshold, and a controller operably coupled to each of the pressure sensor, the cuff pump, and the memory. The controller can be configured to (i) determine, based on the real-time respiration pattern, an inhalation period and an exhalation period of the patient, (ii) operate, during the determined inhalation period, the cuff pump to increase the cuff pressure to the maximum cuff pressure threshold, and (iii) operate, during the determined exhalation period, the cuff pump to decrease the cuff pressure to the minimum cuff pressure threshold.

### CROSS-REFERENCE TO RELATED APPLICATIONS

The present application claims the benefit of U.S. Provisional Patent Application No. 73/760,577, filed February 19, 2025, the disclosure of which is incorporated herein by reference in its entirety.

### TECHNICAL FIELD

The present technology generally relates to medical systems and, in particular, to automated cuff pressure modulation systems for endotracheal tubes.

### BACKGROUND

Tracheal intubation is performed on approximately 13–20 million patients each year in the U.S. alone and is the third most frequently performed procedure in United States hospitals. The procedure involves inserting an endotracheal tube (ETT), connected to a ventilator or other supply of air, through the patient’s nose or mouth into the trachea to open the airway. The procedure can be performed before and/or during airway obstruction, cardiac arrest, apnea, surgery, and/or loss of consciousness (e.g., via anesthesia). Despite its regularity, however, intubation can be a complex and risky procedure, the success of which may depend on preparation, positioning and placement of the ETT, and post-intubation management.

In particular, because of the numerous steps required for and the invasive nature of proper intubation, intubation-related injuries are common. Two regular injuries from ETT use are post-operative sore throat and hoarseness. Also, leakages around the ETT, which can cause improper ventilation, hypoxemia, and pollution from anesthetic mixtures, are common. Incorrect depth placement of ETTs and oral pressure injuries are additional issues that affect a large number of patients. Furthermore, the most common and dangerous issues are tracheal and laryngeal injuries, with laryngeal injuries occurring in 67% of intubation patients. In light of these potential complications, there is a need for improved systems for ETTs and tracheal intubation.

### BRIEF DESCRIPTION OF THE DRAWINGS

Many aspects of the present disclosure can be better understood with reference to the following drawings. The components in the drawings are not necessarily to scale. Instead, emphasis is placed on illustrating clearly the principles of the present disclosure. The drawings should not be taken to limit the disclosure to the specific embodiments shown, but are provided for explanation and understanding.

FIG. 1 is a partially schematic view of a patient’s respiratory system.

FIG. 2 illustrates an endotracheal tube and a cuff.

FIG. 3 is a schematic block diagram of an endotracheal intubation system configured in accordance with various embodiments of the present technology.

FIGS. 4A–4C are schematic block diagrams of various endotracheal intubation systems, each configured in accordance with various embodiments of the present technology.

FIGS. 5A and 5B and front perspective and rear perspective views, respectively, of a cuff pressure modulation device configured in accordance with various embodiments of the present technology.FIG. 6 is an interior view of another cuff pressure modulation device configured in accordance with various embodiments of the present technology.

FIG. 7 is a partially schematic illustration of a disposable kit configured in accordance with various embodiments of the present technology.

FIG. 8A is a graph illustrating endotracheal tube pressure over a respiratory cycle of a patient.

FIG. 8B is a graph illustrating cuff pressure over a respiratory cycle of a patient in accordance with various embodiments of the present technology.

FIGS. 9A and 9B are flowcharts illustrating operation of a cuff pressure modulation device in accordance with various embodiments of the present technology.

FIGS. 10A–10F illustrate various user interfaces configured in accordance with various embodiments of the present technology.

FIG. 11 is a flowchart illustrating a method for modulating cuff pressure in accordance with various embodiments of the present technology.

### DETAILED DESCRIPTION

### I. Overview

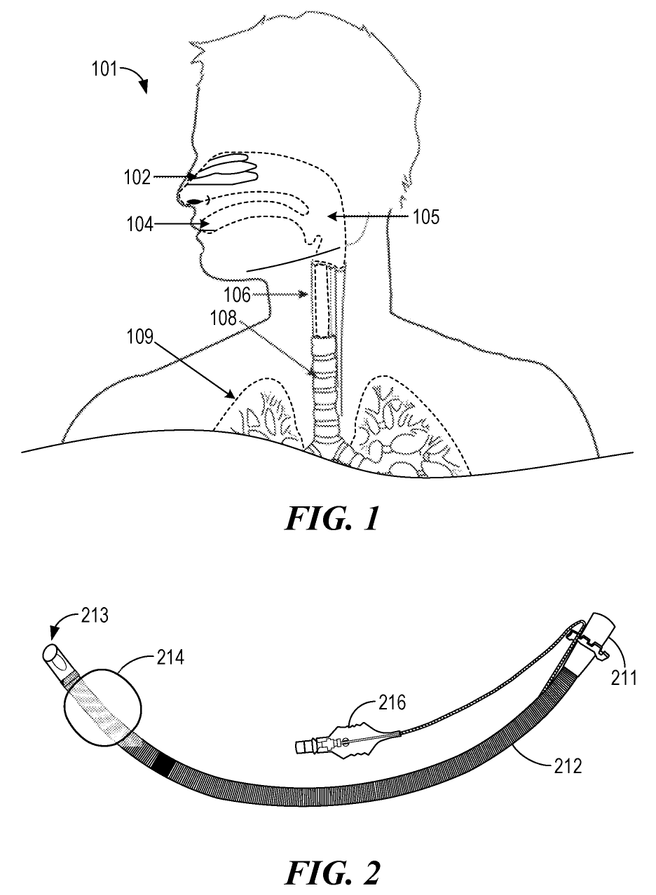

The present technology is generally directed to systems and devices that can automatically modulate cuff pressure for an endotracheal tube (ETT) based on a patient’s respiratory pattern. As shown in FIG. 1, a patient’s respiratory system 91 includes a nasal cavity 92, a mouth 94, a pharynx 95, a larynx 96, a trachea 98, and lungs 99. FIG. 2 illustrates an example ETT 212, which can be a generally elongate tube extending between a proximal end 211 and a distal end 213. A cuff 214 can be a balloon or other inflatable member attached to the ETT 212 near the distal end 213, and can be inflated or deflated via a pilot balloon 216. Notably, the cuff 214 is mechanically coupled to but fluidly disconnected from the ETT 212. The primary function of the cuff 214 is to provide a seal in the trachea 98 to prevent the leakage of gas and thereby ensure proper air delivery via the ETT 212. Another function of the cuff 214 is to keep the ETT 212 centered within the trachea 98 and thereby protect the mucous membranes from the distal end 213, which can be relatively blunt and therefore cause injury.

During a conventional intubation procedure, the distal end 213 of the ETT 212 and the cuff 214 can be inserted into the trachea 98 via the nasal cavity 92 or the mouth 94, the pharynx 95, and the larynx 96. The proximal end 211 of the ETT 212 can be operably coupled to a ventilator (not shown), and the pilot balloon 216 can be operably coupled to a pump (not shown). Thus, air can be delivered to the patient’s lungs 99 via the ETT 212 and the cuff 214 can be inflated at the trachea 98 via the pilot balloon 216. However, because the cuff 214 operates by abutting or pressing against the surrounding tissue walls of the trachea 98, improper cuff pressure can lead to various issues. For instance, cuff pressure that is too high can lead to a decrease in tracheal blood flow, lesions, tracheomalacia, bleeding, tracheal rupture, stenosis, nerve palsy, ischemic necrosis, mucosal damage, and tracheoesophageal fistula. Conversely, cuff pressure that is too low can fail to create a proper seal and lead to inadequate ventilation, oral secretions, and ventilator-associated pneumonia.

Embodiments of the present technology address at least some of the above described issues. For example, embodiments of the present technology include a cuff pressure modulation device that can modulate cuff pressure in real-time. The device can include a motorized pump that can pump air into and out of the cuff in response to control signals. The device can also include a sensor that tracks the patient’s respiratory pattern, such as by measuring the pressure of airflow through the ETT. The trachea naturally expands and contracts during inhalation and exhalation, respectively, so the patient’s respiratory pattern can be a proxy for tracheal movement. Therefore, the device can advantageously expand and contract the cuff in accordance with the patient’s respiratory pattern such that the size of the cuff dynamically tracks the size of the trachea in real-time. In some embodiments, the device measures tracheal movement more directly, such as by measuring cuff pressure.

In some embodiments, prior to modulating cuff pressure, the cuff pressure modulation device can enter a calibration mode in which the device can learn the user’s respiratory pattern. For example, different patients and different ventilators can be associated with different ETT pressures. Calibration can help ensure that the subsequent cuff pressure modulation is appropriate for the specific patient and the specific ventilator in use. In some embodiments, the device allows a physician or other operator to manually set maximum and minimum cuff pressure thresholds, and the subsequent cuff pressure modulation can increase and decrease the cuff pressure between the two thresholds. If the cuff pressure, as measured by another sensor of the device, ever departs from the intended cuff pressure, the device can alert the physician or other operator.

The various features of embodiments of the present technology described herein enable the cuff pressure modulation device to provide increased levels of customization, flexibility, and safety. In particular, by modulating cuff pressure in real-time based on the patient’s respiratory pattern, the device can reduce of the risk of injury to the patient while maintaining a proper seal at the trachea. Moreover, the device can be used as an add-on to existing ventilators, allowing quick and easy adoption.

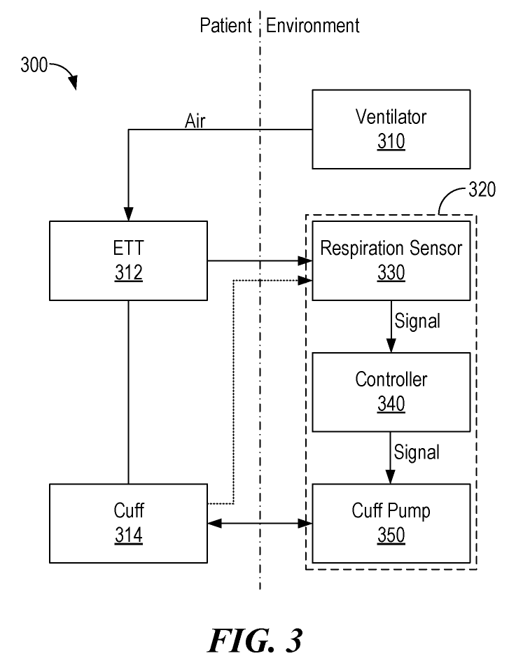

In the following description, specific details are set forth to provide a thorough understanding of aspects of the present technology. One skilled in the relevant art will recognize, however, that the systems, devices, and techniques described herein can be practiced without one or more of the specific details set forth herein, or with other methods, components, materials, etc. The terminology used in the description presented below is intended to be interpreted in its broadest reasonable manner, even though it is being used in conjunction with a detailed description of certain specific embodiments of the present technology. Certain terms may even be emphasized below; however, any terminology intended to be interpreted in any restricted manner will be overtly and specifically defined as such in this Detailed Description section. Additionally, the present technology can include other embodiments that are within the scope of the examples or claims but are not described in detail with respect to FIGS. 3–11.

Reference throughout this specification to an “example” or an “embodiment” means that a particular feature, structure, or characteristic described in connection with the example or embodiment is included in at least one example or embodiment of the present technology. Thus, use of the phrases “for example,” “as an example,” or “an embodiment” herein are not necessarily all referring to the same example or embodiment and are not necessarily limited to the specific example or embodiment discussed. Furthermore, features, structures, or characteristics of the present technology described herein may be combined in any suitable manner to provide further examples or embodiments of the present technology.

Reference numbers used in the figures of the present disclosure follow a numbering convention in which (i) the first digit or digits correspond to the first figure in which a particular element or component is introduced and (ii) the remaining digits identify that particular element or component in the figures. Unless otherwise specified or made clear from context, similar references numbers are used across multiple figures to denote generally similar and/or identical components. For example, reference number 92 can be used to reference an element “2” that was first introduced in FIG. 1. Use of reference number 92 in FIG. 2 can identify the element “2” from FIG. 1 in FIG. 2. Use of reference number 202 in FIG. 2 can be used to reference an element “2” that was first introduced in FIG. 2, and that may (depending on context) be generally similar and/or identical to the element “2” corresponding to reference number 92 that was first introduced in FIG. 1.

It is appreciated that while the discussion below may focus specifically on endotracheal tubes, embodiments of the present technology apply equally to tracheostomy tubes and/or other airway tubes. Also, the headings provided herein are for convenience only and are not to be used to interpret the scope of the claimed technology.

### II. Select Embodiments of an Endotracheal Intubation System

FIG. 3 is a schematic block diagram of an endotracheal intubation system 300 (“the system 300”) configured in accordance with various embodiments of the present technology. The system 300 can include a ventilator 310, an endotracheal tube (ETT) 312, a cuff 314 attached to the ETT 312, and a cuff pressure modulation device 320. The ETT 312 and the cuff 314 can be generally similar to the ETT 212 and the cuff 214 of FIG. 2, respectively. When in use, the ETT 312 and the cuff 314 can be disposed at least partially inside the patient (e.g., in the patient’s trachea), and the ventilator 310 and the cuff pressure modulation device 320 can be positioned in an external environment and near the patient.

The ventilator 310 can be a conventional ventilator or other device that can provide air to the patient via the ETT 312. For example, the ventilator 310 can be coupled to a proximal end of the ETT 312 while the distal end of the ETT 312 is positioned in the patient’s trachea. The ETT 312 can have dimensions suitable for insertion into the patient’s throat via the nose or mouth. For example, the ETT 312 can have an inner diameter between 8.5–8 millimeters (mm). The cuff 314 can be a balloon or other inflatable member attached to the ETT 312 near the distal end thereof.

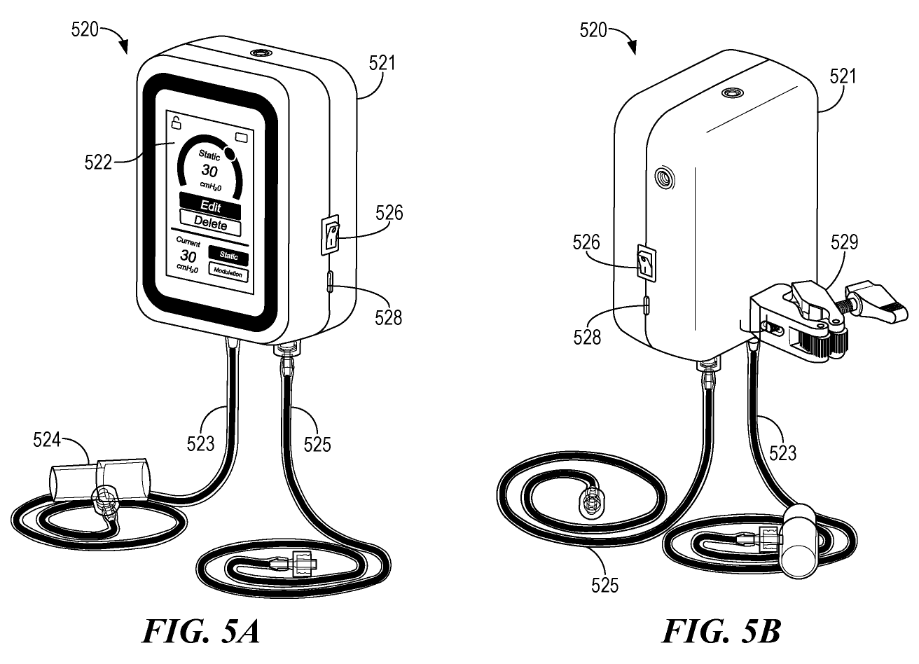

The cuff pressure modulation device 320 can include, among other components, a respiration sensor 330, a controller 340, and a cuff pump 350. The respiration sensor 330 can be operably coupled to (e.g., in fluid communication with) the ETT 312 via a tube or other connection (not shown). The controller 340 can be operably coupled to each of the respiration sensor 330 and the cuff pump 350. The cuff pump 350 can be operably coupled to (e.g., in fluid communication with) the cuff 314 via a tube or other connection (not shown). In operation, the respiration sensor 330 can determine a respiratory parameters of the patient. For example, the respiration sensor 330 can be pressure sensor that can measure the pressure of airflow delivered through the ETT 312, and generate corresponding sensor output signals. The controller 340 can receive the sensor output signals from the respiration sensor 330 and generate control signals for the cuff pump 350. The cuff pump 350 can pump a fluid (e.g., air) into and out of the cuff 314 in response to the control signals generated by the controller 340. Example cuff pressure modulation devices are illustrated in and described below with reference to FIGS. 5A–7.

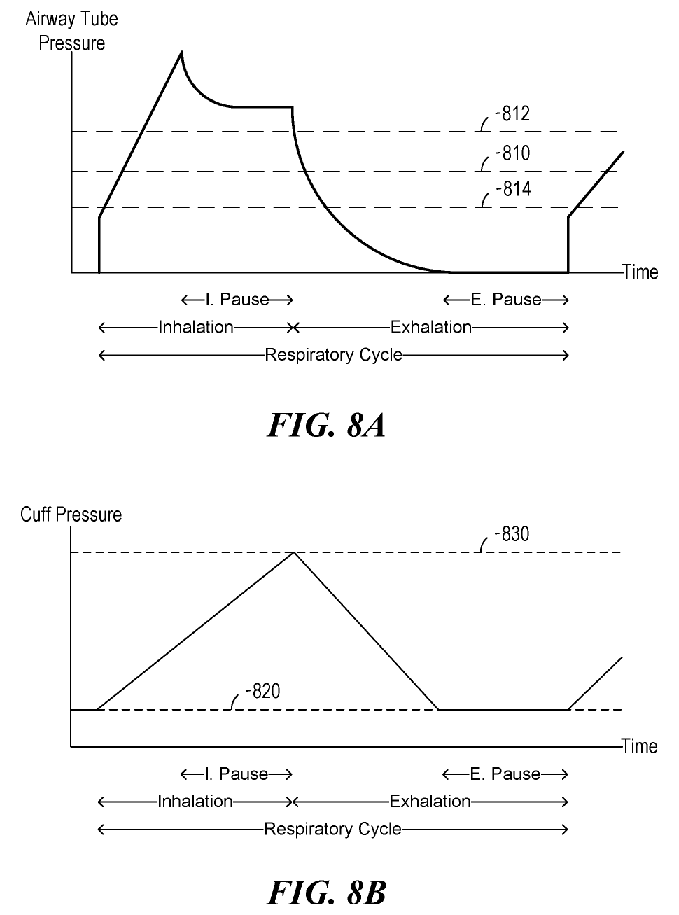

In some embodiments, the controller 340 determines the patient’s respiratory pattern based on the sensor output signals from the respiration sensor 330, and generates the control signals based on the determined respiratory pattern. For example, the controller 340 can gauge the timings of the patient’s inhalation and exhalation, and instruct the cuff pump 350 to increase the cuff pressure during inhalation and decrease the cuff pressure during exhalation. The controller 340 can determine such timings on a real-time basis and/or on a predictive basis. When determining on a real-time basis, the controller 340 can detect instantaneous changes in the ETT pressure (e.g., indicating a switch from exhalation to inhalation) and generate the control signals immediately thereafter. When determining on a predictive basis, the controller 340 can use historical records of one or more respiratory cycles of the patient to predict the timings of one or more subsequent respiratory cycles. Example operation of a cuff pressure modulation device is illustrated in and described below with reference to FIGS. 8A–11.

It is appreciated that the system 300 of FIG. 3 is merely a representative example, and that in other embodiments, additional, fewer, and/or alternative components can be included. For example, the ventilator 310 can be omitted and the patient can be intubated while breathing atmospheric or room air (e.g., by the patient’s own breathing). As another example, while FIG. 3 specifically illustrates an endotracheal intubation system, embodiments of the present technology are not limited so (e.g., can be used in tracheostomies).

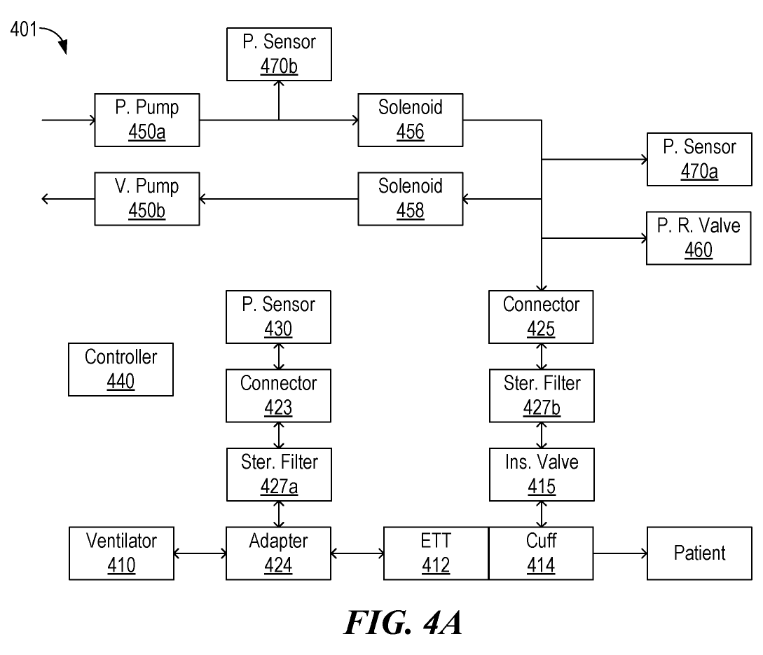

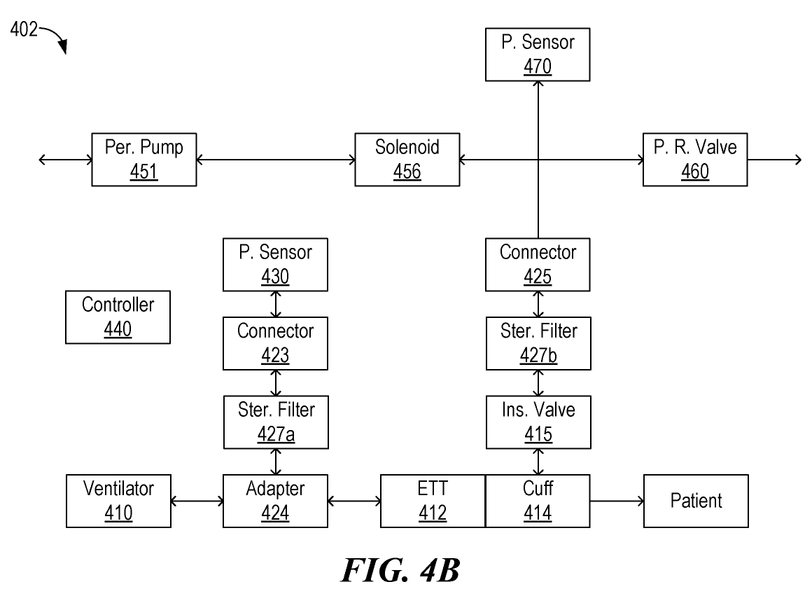

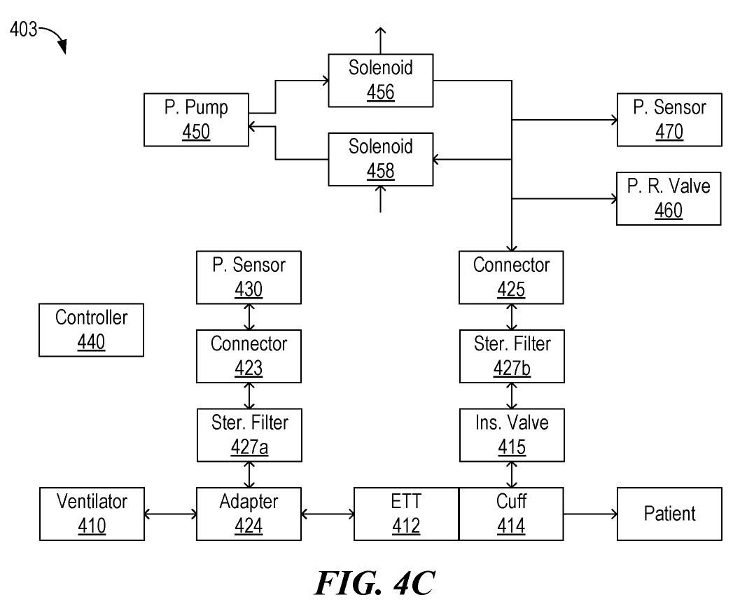

FIGS. 4A–4C are schematic block diagrams of various endotracheal intubation systems 401, 402, 403 (collectively referred to as “the systems 401, 402, 403”), each configured in accordance with various embodiments of the present technology. Each of the systems 401, 402, 403 can be an example of the system 300 of FIG. 3. In FIGS. 4A–4C, each of the systems 401, 402, 403 can include a ventilator 410, an ETT 412, a cuff 414, and a controller 440. The ETT 412 and the cuff 414 can be disposed in the respiratory tract of a patient. The controller 440 can be used to operate the various components described below. The ventilator 410 can be coupled to the ETT 412 via an adapter 424, which can be a T-valve or other component that can fluidly connect the ventilator 410, the ETT 412, and a pressure sensor 430. The adapter 424 can be coupled to the pressure sensor 430 via a connector 423 (e.g., a disposable tube), which can include a sterile filter 427a. The cuff 414 can be operably connected to a connector 425, which can include a sterile filter 427b and an insufflation valve 415.

Referring first to FIG. 4A, the connector 425, operably coupled to the cuff 414, can be operably (e.g., fluidly) coupled to each of a pressure pump 450a, a vacuum pump 450b, a pressure relief valve 460, and a pressure sensor 470a. In particular, the connector 425 can be coupled to the pressure pump 450a via a first solenoid valve 456 (or other valve or switch), and to the vacuum pump 450b via a second solenoid valve 458 (or other valve or switch). Also, another pressure sensor 470b can be coupled to pressure the pressure provided by the pressure pump 450a, such as by coupling the pressure sensor 470b to each of the pressure pump 450a and the first solenoid valve 456 via a T-valve.

Referring next to FIG. 4B, the connector 425, operably coupled to the cuff 414, can be operably (e.g., fluidly) coupled to each of a peristaltic pump 451, the pressure relief valve 460, and the pressure sensor 470. In particular, the connector 425 can be coupled to the peristaltic pump 451 via the solenoid valve 456 (or other valve or switch).

Referring next to FIG. 4C, the connector 425, operably coupled to the cuff 414, can be operably (e.g., fluidly) coupled to each of a pressure pump 450, the pressure relief valve 460, and the pressure sensor 470. In particular, the connector 425 can be coupled to the pressure pump 450 via the first solenoid valve 456 (or other valve or switch) and the second solenoid valve 458 (or other valve or switch) along two separate paths.

Referring to FIGS. 4A–4C together, in operation, the pumps 450a, 450b, 451, 450 can provide discharge and/or suction of a fluid (e.g., air) to inflate or deflate the cuff 414. The solenoid valves 456, 458 can help ensure that the discharge and suction are provided at desired rates. The pressure relief valve 460 can selectively provide an outlet in case the pressure provided by the pumps exceeds a threshold, and the pressure sensors 470a, 470b, 470 can monitor the cuff pressure. Additional details regarding operation of each of the components described above are provided further below.

FIGS. 5A and 5B and front perspective and rear perspective views, respectively, of a cuff pressure modulation device 520 configured in accordance with various embodiments of the present technology. The cuff pressure modulation device 520 can be an example of the cuff pressure modulation device 320 of FIG. 3, and can include a housing 521, a display screen 522 (FIG. 5A), a power switch 526, a charging port 528, and a coupler 529 (FIG. 5B). The cuff pressure modulation device 520 can also include a pair of ports for connecting to an ETT connector 523 and a cuff connector 525.

In the illustrated embodiment, the housing 521 has a generally prismatic form factor for storing various electronics, pumps, actuators, and/or the like therein. The housing 521 can be made of plastic, wood, metal, and/or other suitable material, and can have a size suitable for portability. For example, the housing 521 can have dimensions no greater than 9” × 7” × 3”. The power switch 526 and the charging port 528 can be disposed on the same side or different sides of the housing 521. The power switch 526 can be used to turn on or off the cuff pressure modulation device 520, and the charging port 528 (e.g., a USB-C port) can be used to charge the cuff pressure modulation device 520. The coupler 529 (e.g., a manually operable clamp) can be disposed on a rear side of the housing 521, and can be used to secure and support the cuff pressure modulation device 520 onto another object (e.g., a ventilator rack).

The display screen 522 can be disposed on a front side of the housing 521, and may or may not be a touchscreen. FIG. 5A shows the display screen 522 displaying a user interface including a touch-lock button, a battery level indicator, an operating mode of the cuff pressure modulation device 520, a target cuff pressure, a button for adjusting the target cuff pressure, a button for deflating the cuff, a current cuff pressure, and buttons for switching the operating mode of the cuff pressure modulation device 520. It is appreciated that the illustrated user interface is merely a representative example, and that the display screen 522 can display other user interfaces with fewer, additional, and/or alternative user options.

The ETT connector 523 and the cuff connector 525 can be operably coupled to the cuff pressure modulation device 520 via ports on, for example, a bottom side of the housing 521. As discussed in greater detail below with reference to FIG. 6, the ETT connector 523 can fluidly couple an ETT (e.g., the ETT 312 of FIG. 3) to the cuff pressure modulation device 520, and the cuff connector 525 can couple a cuff (e.g., the cuff 314) to the cuff pressure modulation device 520. In some embodiments, the ETT connector 523 can be fluidly connected to the ETT via a T-valve 524. For example, the ETT connector 523 can be connected to the common port of the T-valve 524, and the two side ports of the T-valve 524 can be connected to the ETT and a ventilator. The T-valve 524 can also be connected to other airway tubes, such as a tracheostomy tube. In some embodiments, the cuff connector 525 can be fluidly connected to a pilot balloon (e.g., the pilot balloon 216 of FIG. 2).

FIG. 6 is an interior view of another cuff pressure modulation device 620 configured in accordance with various embodiments of the present technology. The cuff pressure modulation device 620 can be an example of the cuff pressure modulation device 520 of FIG. 5. The cuff pressure modulation device 620 can include, among other components, a housing 621, a respiration sensor 630, one or more controllers 640, a cuff pump 650, a pressure relief valve 660, and a cuff sensor 670.

The housing 621 can be generally similar to the housing 521 of FIG. 5. For example, the housing 621 can be fitted with a power switch 626, a charging port 628, and ports for connecting to an ETT connector 623 and a cuff connector 625. The housing 621 can enclose and protect the various components of the cuff pressure modulation device 620 described below. The respiration sensor 630 (e.g., a pressure sensor) can be operably coupled to (e.g., in fluid communication with) the ETT connector 623 via an adapter tube 632. Thus, the respiration sensor 630 can measure, for example, the pressure of the airflow in the associated ETT, which corresponds to the patient’s respiration. In some embodiments, the respiration sensor 630 is not positioned inside the housing 621 and is instead positioned elsewhere, such as in the ETT connector 623.

The cuff pump 650 (e.g., a pressure/vacuum DC pump, a peristaltic pump) can be operably coupled to a pump motor 652 that can control operation thereof. The cuff pump 650 can be operably coupled to (e.g., in fluid communication with) the cuff connector 625, and can provide either discharge or suction of a fluid (e.g., air) thereto or therefrom. For example, the cuff pump 650 can include a discharge port 651a and a separate suction port 651b (collectively referred to as “the ports 651”). A pump distributor 654 can fluidly connect both ports 651 to an inlet/outlet distributor 672 operably coupled to the cuff connector 625, but along two separate fluid flow paths. Along the first path, also referred to herein as “the discharge path,” a discharge valve 656 (e.g., a solenoid valve) can control the flowrate of the fluid from the discharge port 651a to the inlet/outlet distributor 672. Along the second path, also referred to herein as “the suction path,” a suction valve 658 (e.g., a solenoid valve) can control the flowrate of the fluid from the inlet/outlet distributor 672 to the suction port 651b.

In the illustrated embodiment, the discharge path is also fluidly connected to the pressure relief valve 660 via a distributor 662 and a pressure relief valve adapter 664. The inlet/outlet distributor 672 can also be fluidly connected to the cuff sensor 670 (e.g., a pressure sensor). The inlet/outlet distributor 672 can, for example, maintain separate fluid flow paths from the cuff connector 625 to each of the discharge port 651a, the suction port 651b, and the cuff sensor 670.

The controllers 640 can be operably coupled to each of the pump motor 652, the discharge valve 656, the suction valve 658, the respiration sensor 630, and the cuff sensor 670. The controllers 640 can include one or more memories (e.g., volatile memory, non-volatile memory) and one or more processors for executing instructions stored on the memories. Other components that may be included, depending on the computational requirements, include dedicated mathematical processors, digital signal processors (DSPs), field-programmable gate arrays (FPGAs), and/or machine learning integrated circuits (such as neural processing units or tensor processing units). The controllers 640 can be operably coupled to a battery 642, which can provide power to not only the controllers 640, but also to the various components operably coupled to the controllers 640. The battery 642 can be charged, e.g., via the charging port 628. Accordingly, the cuff pressure modulation device 620 can run on either battery or grid power. In some embodiments, the cuff pressure modulation device 620 also includes a boost converter that can provide a voltage step-up from the batter 642. The controllers 640 can also be operably coupled to additional components not shown in FIG. 6, such as the display screen 522 of FIG. 5A.

In operation, the controllers 640 can receive sensor output signals from the respiration sensor 630 to determine a respiratory pattern of the patient. For example, based on the pressure readings of the associated ETT, the controllers 640 can determine the timings of the patient’s inhalations and exhalations in real-time. Additional details on interpreting sensor output signals from a respiration sensor are discussed below with reference to FIG. 8A. The controllers 640 can also operate the pump motor 652, the discharge valve 656, and/or the suction valve 658 to provide either discharge or suction of a fluid to or from the cuff connector 625, and thus to or from the associated cuff. For example, the controllers 640 can operate the pump motor 652 and open the discharge valve 656 during the patient’s inhalation to inflate the cuff, and operate the pump motor 652 and open the suction valve 658 during the patient’s exhalation to deflate the cuff. In some embodiments, the cuff pump 650 can adjust the cuff pressure to any pressure value between 0–100 cmH₂O. The discharge valve 656 and the suction valve 658 may be operably tied such that one is closed while the other is open. Also, the inlet/outlet distributor 672 can help ensure that discharge and suction are provided to the cuff separately. Additional details on pumping a cuff based on a respiratory pattern of the patient are discussed below with reference to FIG. 8B.

While providing discharge, if the pressure increase provided by the cuff pump 650 exceeds a predetermined threshold, the pressure relief valve 660 can automatically release some of the fluid (e.g., air) into the interior of the housing 621. This can help avoid inadvertently over-pressurizing and excessively inflating the cuff, which can be dangerous to the patient. The housing 621 can include one or more vents to prevent over-pressurization of the housing 621. Additional safety mechanisms that the cuff pressure modulation device 620 can incorporate include maximum pressure limits with automatic shutoff, watchdog timers to detect system faults, sensor validation algorithms to detect sensor failures or disconnections, a failsafe deflation protocol that completely deflates the cuff when safety thresholds are exceeded or system faults are detected, etc. Moreover, the cuff pump 650, the discharge valve 656, and/or the suction valve 658 can be implemented such that their default, unpowered states result in no pressure application to the cuff, providing passive safety.

The cuff sensor 670 can monitor the inflation pressure inside the cuff and thus detect any unacceptable deviations from the desired cuff pressure. Also, in some embodiments, the controllers 640 use sensor output signals from the cuff sensor 670 to determine a respiratory pattern of the patient. For example, as the trachea expands and contracts during respiration, the trachea places pressure on the cuff, which can be detected by the cuff sensor 670. Accordingly, the cuff sensor 670 can not only serve as part of a safety mechanism that detects and alerts operators of unsafe and/or error conditions, but also replace or supplement the respiration sensor 630 for measuring airflow in the ETT and determining timings of the patient’s inhalations and exhalations.

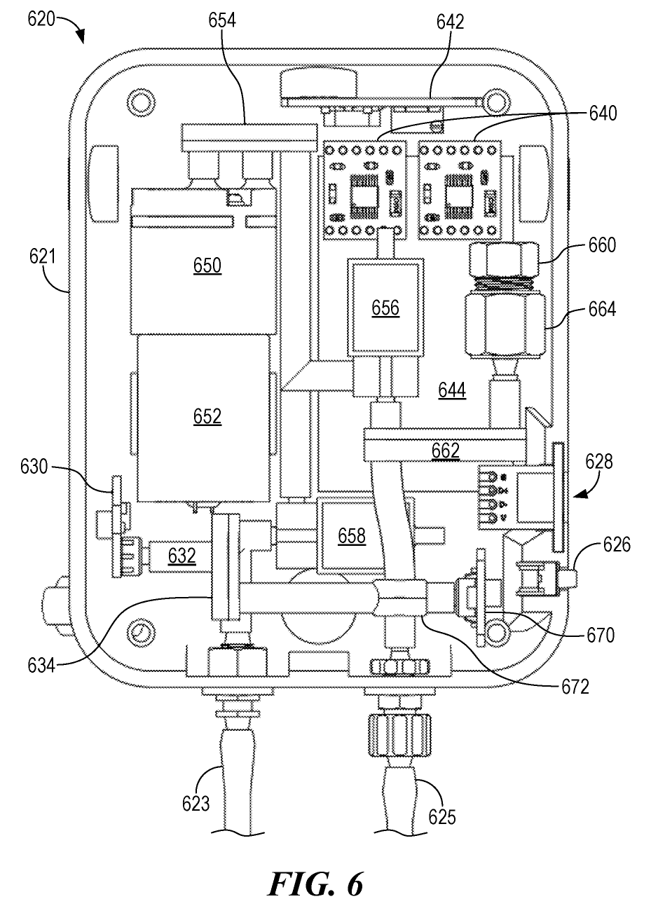

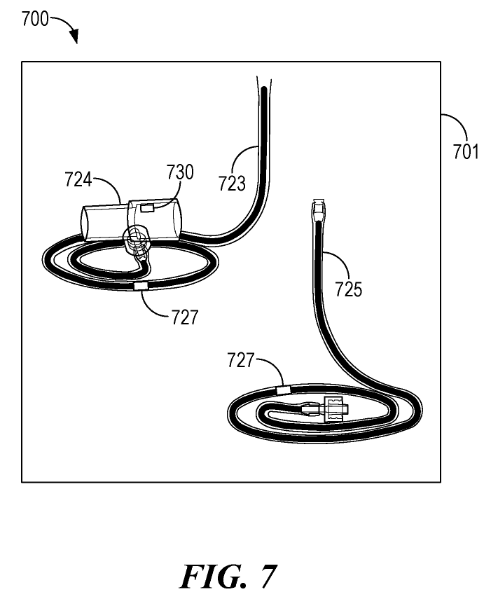

FIG. 7 is a partially schematic illustration of a disposable kit 700 configured in accordance with various embodiments of the present technology. The disposable kit 700 can include a container 701, an ETT connector 723, and a cuff connector 725. The container 701 can be a box, a bag (e.g., a plastic bag), and/or other suitable medical packaging for sterile objects. The ETT connector 723 and the cuff connector 725 can be examples of the ETT connector 523 and the cuff connector 525 of FIG. 5, respectively. For example, the ETT connector 723 can be fitted with a T-valve 724 for connecting to both a ventilator and an ETT or other respiratory-assistance tube, such as a tracheostomy tube.

The disposable kit 700 can be a single-use kit designed to be used with a particular patient, then discarded. Also, the components of the disposable kit 700 (e.g., the ETT connector 723 and the cuff connector 725) can be compatible with a cuff pressure modulation device of the present technology. For example, the cuff pressure modulation device 620 of FIG. 6 can be a reusable component designed to be used with multiple patients and/or multiple times, and the components of the disposable kit 700 can be coupled to the cuff pressure modulation device 620 temporarily during use, then discarded and swapped for another disposable kit 700 for a different patient. One or more of the components of the disposable kit 700 (e.g., including the container 701) can be recyclable, biodegradable, and/or the like.

In some embodiments, the ETT connector 723 is fitted with a respiration sensor 730. In the illustrated embodiment, for example, the respiration sensor 730 is disposed in the T-valve 724, but in other embodiments, the respiration sensor 730 can be disposed elsewhere in or on the ETT connector 723. The respiration sensor 730 can be operably coupleable to a cuff pressure modulation device via a wireless connection or a wired connection (not shown). In operation, the respiration sensor 730 can supplement or replace the functionality of the respiration sensor 630 of FIG. 6 and monitor the ETT pressure. Integrating the respiration sensor 730 with the ETT connector 723 can provide a closer-to-source (and thus potentially more accurate and/or less delayed) pressure reading. In some embodiments, the ETT connector 723 and/or the cuff connector 725 include filters 727 (e.g., moisture filters, dust filters).

It is appreciated that the illustrated embodiments of FIGS. 5A–8 are merely illustrative examples. For example, while FIG. 6 illustrates the various components of the cuff pressure modulation device as enclosed in a singular housing, in other embodiments, the various components can be distributed in two or more separate enclosures or devices. As another example, while FIG. 7 illustrates the container 701 as a singular packaging for both the ETT connector 723 and the cuff connector 725, in other embodiments, separate containers can be used to package the two connectors 723, 725.

### III. Select Embodiments of a Method for Modulating Cuff Pressure

FIG. 8A is a graph illustrating endotracheal tube pressure over a respiratory cycle of a patient. The respiratory cycle can be divided into an inspiration or inhalation period and an expiration or exhalation period. At the start of the inhalation period, as the patient breathes in, the pressure of airflow in an airway tube (e.g., an ETT) can rise as shown. A latter portion of the inhalation period, however, can be marked by a decrease in airway tube pressure, corresponding to an inspiratory pause. At the start of the exhalation period, as the patient breathes out, the airway tube pressure can continue to decrease as shown. A latter portion of the exhalation period, however, can be marked by a constant airway tube pressure, corresponding to an expiratory pause. An average respiratory cycle can include, for example, 1–3 seconds of inhalation, then 1–3 seconds of exhalation, followed by the expiratory pause. It is appreciated that the ETT pressure graph of FIG. 8A is merely an illustrative example, and that embodiments of the present technology can work with various respiratory patterns (e.g., eupnea, hyperpnea, bradypnea, tachypnea, apnea, Cheyne-Stokes, Biot, Kussmaul) which can vary greatly depending on the specific patient.

As discussed above with reference to FIG. 6, the respiration sensor 630 can monitor the airway tube pressure (e.g., the ETT pressure) in real-time. Therefore, by analyzing parameters in the airway tube pressure, the controllers 640 can determine the timings of the inhalation period and the exhalation period. For example, the controllers 640 can determine a baseline or average airway tube pressure 810 over time (e.g., during an initial calibration period) and use the average airway tube pressure 810 as a threshold between inhalation and exhalation. Specifically, the controllers 640 can (i) inflate the cuff if the current airway tube pressure is above the average airway tube pressure 810 or (ii) deflate the cuff if the current airway tube pressure is below the average airway tube pressure 810. In other embodiments, the controllers 640 can determine an upper threshold 812 and a lower threshold 814, determine that the patient is inhaling when the airway tube pressure is above the upper threshold 812, and determine that the patient is exhalating when the airway tube pressure is below the lower threshold 814. Other baseline thresholds can be determined. Alternatively, the controllers 640 can analyze rises, falls, and/or other parameters of the airway tube pressure to distinguish between inhalation and exhalation. The controllers 640 can also identify the inspiratory and expiratory pauses and pump the cuff differently during such pauses (e.g., keep the cuff at a constant pressure during the expiratory pause).

Determination of the patient’s respiratory pattern can be performed on a real-time basis or on a predictive basis. When performed on a real-time basis, the controllers 640 can inflate or deflate the cuff based on the instantaneous, real-time airway tube pressure reading (e.g., how it compares to the airway tube pressure 810, whether it is increasing or decreasing). When performed on a predictive basis, the controllers 640 can keep a record of the airway tube pressure over one or more respiratory cycles, then inflate or deflate the cuff based on an anticipated or predicted airway tube pressure reading. In some embodiments, the determination of the patient’s respiratory pattern is a combination of both bases (e.g., using an average of the two, using one as a safety check).

FIG. 8B is a graph illustrating cuff pressure over a respiratory cycle of a patient in accordance with various embodiments of the present technology. As shown, the controllers 640 can inflate or deflate the cuff according to timings that generally align with the patient’s inhalation and expiration. In the illustrated embodiment, the cuff pressure is increased linearly from a minimum cuff pressure threshold 820 to a maximum cuff pressure threshold 830 during a period generally corresponding to the patient’s inhalation (e.g., as determined by the analysis discussed above with reference to FIG. 8A) and is decreased linearly during a period generally corresponding to the patient’s exhalation. In particular, the cuff pressure is maintained at the minimum cuff pressure threshold 820 during a period generally corresponding to the patient’s expiratory pause. Accordingly, the cuff can inflate and expand as the patient’s trachea expands, and deflate and contract as the patient’s trachea contracts.

It is appreciated that the cuff pressure graph of FIG. 8B is merely an illustrative example, and that other embodiments of the present technology can modulate cuff pressure in different ways. For example, instead of increasing and decreasing linearly, cuff pressure can increase or decrease along non-linear curves over time. As another example, the cuff pressure may not be maintained at a constant cuff pressure during the period generally corresponding to the patient’s expiratory pause (e.g., the cuff pressure may continue to decrease but at a slower rate than illustrated).

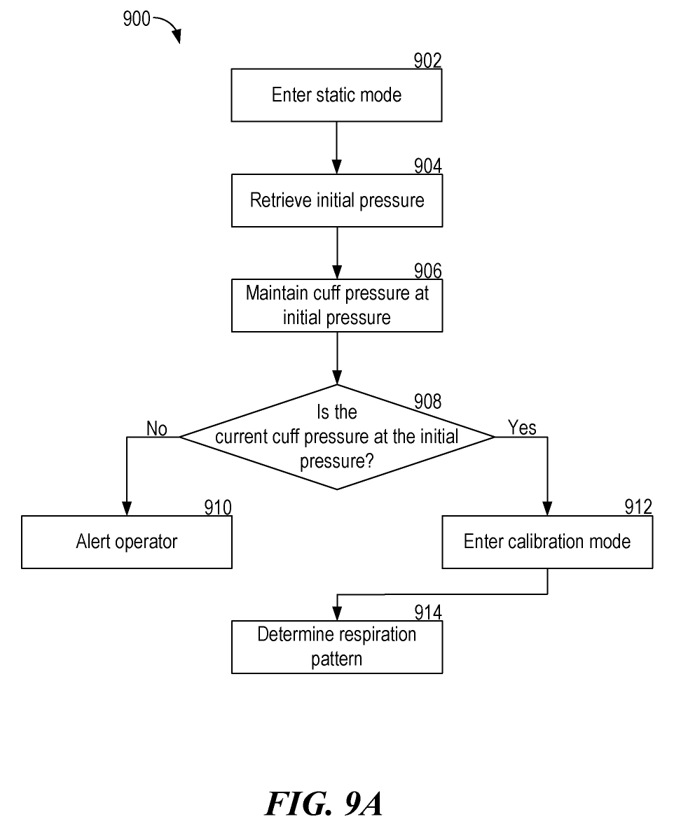

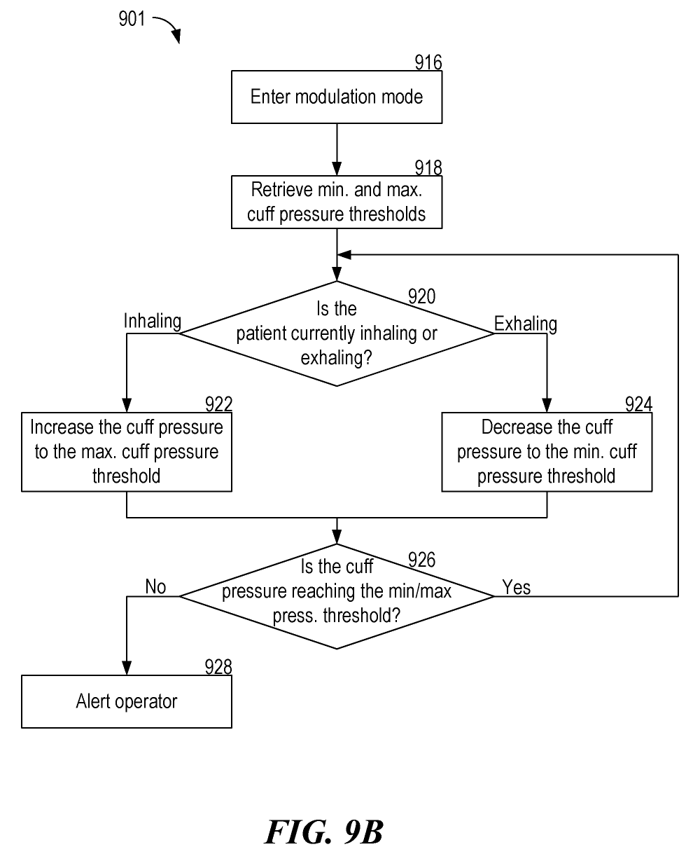

FIGS. 9A and 9B are flowcharts 900, 901 illustrating operation of a cuff pressure modulation device in accordance with various embodiments of the present technology. Referring first to the flowchart 900 of FIG. 9A, at block 902, a cuff pressure modulation device (e.g., the cuff pressure modulation device 620 of FIG. 6) can enter a static mode. While in the static mode, the cuff pressure modulation device can proceed to block 904 and retrieve an initial pressure (e.g., from a memory thereof). The initial pressure can be input by an operator and stored in a memory of the cuff pressure modulation device. For example, the cuff pressure modulation device can prompt the operator to enter the initial pressure via a user interface (e.g., the display screen 522 of FIG. 5) and/or receive the initial pressure via data transfer. Alternatively, the cuff pressure modulation device can prompt the user to approve using the most recently used initial pressure. At block 906, the cuff pressure modulation device can inflate the cuff and maintain the cuff pressure at the retrieved initial pressure. In particular, while in the static mode, the cuff pressure modulation device can function as a conventional automated cuff controller that detects cuff pressure changes (e.g., due to patient movement, a gas leak) and pumps air in or out as needed. The initial pressure can serve as a constant pressure that the cuff can be inflated to before modulation. The initial pressure can be between 0–100 cmH₂O or between 20–30 cmH₂O, such as about 25 cmH₂O. For example, the device can prompt the user to input an initial pressure value that is specifically between 0 cmH₂O and 100 cmH₂O, display an error message if the user enters a value lower than 0 cmH₂O or higher than 100 cmH₂O, and/or the like.

The cuff pressure modulation device can then proceed to block 908 to determine whether the current cuff pressure (e.g., measured by the cuff sensor 670 of FIG. 6) is at the initial pressure. In the event that the current cuff pressure is not at the initial pressure, the cuff pressure modulation device can, at block 910, alert the operator. The fact that the cuff pressure is not at the set pressure can indicate one or more error conditions, such as an improper connection, a leak, and/or the like. Accordingly, the cuff pressure modulation device can halt operations to avoid causing patient complications. In the event that the current cuff pressure is at the initial pressure, the cuff pressure modulation device can, at block 912, enter a calibration mode. The cuff pressure modulation device can switch to the calibration mode in response to a user input, a lapse of a predetermined time period, and/or the like. Once in the calibration mode, the cuff pressure modulation device can establish baseline pressure readings to help account for differences between different patients and/or different respiratory assistance environments such as different ventilators, the ambient temperature and elevation which can affect absolute pressure measurements, etc. In particular, the cuff pressure modulation device can calibrate to patients regardless of whether they are intubated with a ventilator or they are breathing atmospheric or room air.

The cuff pressure modulation device can proceed to block 914 to determine a respiration pattern of the patient. For example, the respiration sensor 630 can track the airway tube pressure over one or more respiration cycles and determine an average airway tube pressure (e.g., the average airway tube pressure 810 of FIG. 8A), airway tube pressure thresholds (e.g., the upper threshold 812 and the lower threshold 814), a maximum airway tube pressure, a minimum airway tube pressure, and/or the like. The cuff pressure modulation device may also track the timing of the respiration pattern to determine, e.g., the duration of an average respiration cycle. The calibration can consider at least one, two, three, four, five, six, seven, eight, nine, ten, or more respiratory cycles and last at least 1, 2, 3, 4, 5, 6, 7, 8, 9, 10, 15, 20, 25, 30, or more seconds, and/or no more than 30 seconds, 1 minute, 1 minute and 30 seconds, 2 minutes, or more. In some embodiments, the determined respiration pattern is communicated to the operator. For example, the cuff pressure modulation device can display, on the display screen 522, various ETT values, durations, and/or the like.

Referring next to the flowchart 901 of FIG. 9B, the flowchart 901 can be a continuation of the flowchart 900 of FIG. 9A. At block 916, the cuff pressure modulation device can enter a modulation mode. The cuff pressure modulation device can switch to the modulation mode in response to a user input, a lapse of a predetermined time period, termination of the calibration process, and/or the like. Once in the modulation mode, the cuff pressure modulation device can proceed to block 918 to retrieve a minimum cuff pressure threshold and a maximum cuff pressure threshold (e.g., from a memory thereof). In some embodiments, the minimum and maximum cuff pressure thresholds are input by the operator (e.g., in response to the respiration pattern communicated to the operator after block 914 of FIG. 9A). In other embodiments, the cuff pressure modulation device automatically determines the minimum and maximum cuff pressure thresholds based on, e.g., the respiration pattern determined at block 914. For example, [cuff sensor] [respiration sensor] “optimal” The automatic determination can be based on a mathematical formula, an artificial intelligence model trained to determine such thresholds based on a given respiration pattern, and/or the like. Each of the minimum cuff pressure threshold and the maximum cuff pressure threshold can be between, e.g., 0–100 cmH₂O (e.g., about 21 cmH₂O, about 28 cmH₂O).

The cuff pressure modulation device can proceed to block 920 to determine whether the patient is currently inhaling or exhaling. The determination can be based on the real-time airway tube pressure (e.g., as measured by the respiration sensor 630). For example, the cuff pressure modulation device can use (i) rising and falling edge detection in the measured airway tube pressure to establish trending changes in breathing rate, (b) regression-based curve fitting to fit measured data to predetermined models of breathing patterns, accommodating both voluntary breathing and positive pressure-driven ventilation, (c) least-squares fitting as a curve-fitting approach to minimize the squared differences between model predictions and input sensor data, and/or (d) a machine learning approach to dynamically adjust the predictive model based on real-time input data, enabling patient-specific optimization.

Additionally or alternatively, the cuff pressure modulation device can determine whether the patient is inhaling or exhaling based on readings of, e.g., the cuff sensor 670 of FIG. 6. For example, if the real-time cuff pressure is increasing, this may indicate that the trachea is contracting and thus the patient is exhaling. Conversely, if the real-time cuff pressure is decreasing, this may indicate that the trachea is expanding and thus the patient is inhaling. In some embodiments, the cuff pressure modulation device uses multi-variate models that combine data inputs from both the respiration sensor 630 and the cuff sensor 670 to enable predictive inflation and deflation adjustments.

In the event that the patient is inhaling, the cuff pressure modulation device can proceed to block 922 to increase the cuff pressure to the maximum cuff pressure threshold. This can help compensate for the fact that the trachea expands during inhalation. In the event that the patient is exhaling, the cuff pressure modulation device can proceed to block 924 to decrease the cuff pressure to the minimum cuff pressure threshold. This can help compensate for the fact that the trachea contracts during exhalation. The manner in which and/or the rate at which the cuff pressure is increased or decreased can depend on various factors, as discussed above with reference to FIG. 8B.

The cuff pressure modulation device can perform a safety check by continuing to block 926 to determine whether the cuff pressure is reaching the minimum or maximum cuff pressure threshold. In the event that the cuff pressure is not, the cuff pressure modulation device can proceed to block 928 and alert the operator. The fact that the cuff pressure is not at the set pressure can indicate one or more error conditions, such as an improper connection, a leak, and/or the like. As discussed above with respect to block 910, alerting the operator can be important to prevent patient complications. In the event that the cuff pressure is reaching the minimum or maximum cuff pressure threshold, the cuff pressure modulation device can continue operating by returning to block 920 and modulating the cuff pressure based on the patient’s respiration.

In some embodiments, the cuff pressure modulation device can continuously or routinely calibrate while modulating the cuff pressure. For example, instead of using the baseline pressure values (e.g., upper pressure threshold, lower pressure threshold, average pressure level) determined during the calibration process for the entirety of the modulation, the cuff pressure modulation device can update those values as the patient breathes during cuff pressure modulation. In such embodiments, the cuff pressure modulation device may automatically or prompt the user to adjust the minimum and/or the maximum cuff pressure thresholds based on the updated baseline pressure values.

It is appreciated that the cuff pressure modulation described herein is performed in real-time based on real-time measurements of the airway tube pressure, the cuff pressure, and/or the like. In “real-time,” as used herein, means that the response time is no more than 1 second, 0.75 seconds, 0.5 seconds, 0.25 seconds, 0.2 seconds, 0.15 seconds, 0.1 seconds, or between 0.1–1 second (or any range therein).

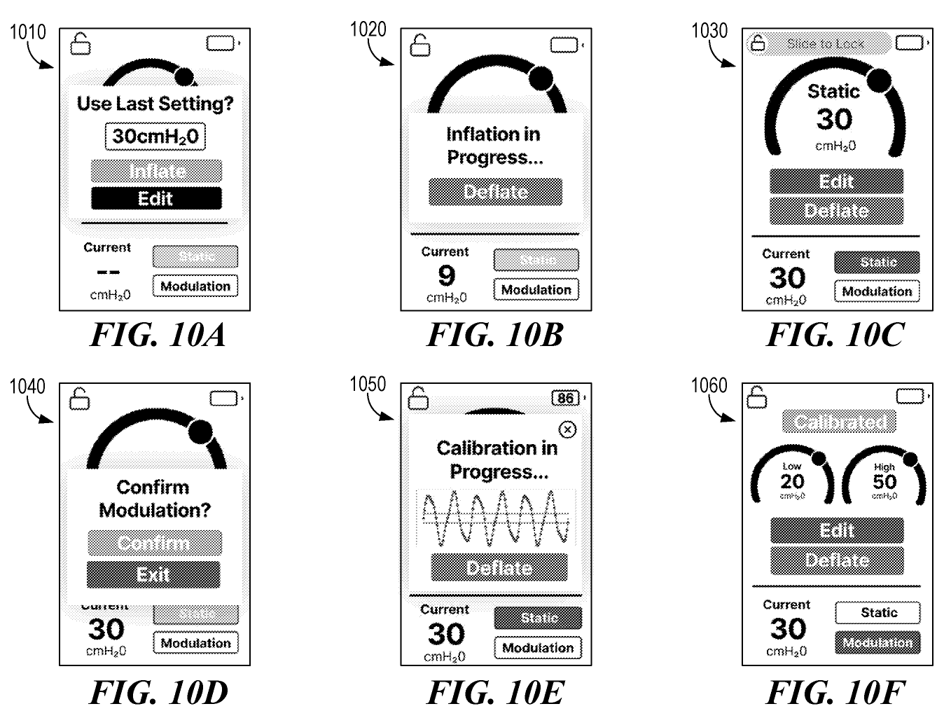

FIGS. 10A–10F illustrate various user interfaces (UIs) 1010, 1020, 1030, 1040, 1050, 1060 configured in accordance with various embodiments of the present technology. Each of the UIs 1010, 1020, 1030, 1040, 1050, 1060 can be an example user interface that can be displayed on the display screen 522 of FIG. 5A.

Referring first to FIG. 10A, the UI 1010 can be displayed upon the cuff pressure modulation device entering static mode (e.g., block 902 of FIG. 9A). As shown, the UI 1010 can prompt the user to select the initial cuff pressure by either selecting the setting used in the past (the user can select “Inflate”) or entering a different setting (the user can select “Edit”) (e.g., block 904 of FIG. 9B).

Referring next to FIG. 10B, the UI 1020 includes a notification indicating that the device is currently inflating the cuff (e.g., to the selected initial cuff pressure). The bottom-left corner of the UI 1020 can display the current cuff pressure as measured by a cuff sensor (“9 cmH₂O”). As shown, the UI 1020 can also include an option to deflate the cuff at any time. For example, the patient may indicate discomfort or pain during the cuff inflation, in which case the user can select “Deflate” to immediately cease inflating and deflate the cuff.

Referring next to FIG. 10C, the UI 1030 displays the current mode (“Static”) and the current cuff pressure (“30 cmH₂O”). The UI 1030 also displays options for the user to adjust the constant cuff pressure (“Edit”) or deflate the cuff (“Deflate”). Moreover, the top of the UI 1030 includes an option for the user to lock the rest of the UI to, for example, prevent accidental adjustment of the cuff pressure. The bottom of the UI indicates whether the device is in the static mode or modulation mode (e.g., by highlighting the corresponding button) and allows user to exit the static mode and begin modulating the cuff pressure by clicking on the corresponding button (“Modulation”).

Referring next to FIG. 10D, the UI 1040 can be displayed in response to the user selecting the “Modulation” option, prompting the user to confirm their selection.

Referring next to FIG. 10E, the UI 1050 can be displayed in response to the user confirming their selection of the “Modulation” option. In particular, the device is in the calibration mode (e.g., block 912 of FIG. 9A) in which the device can determine a real-time respiration pattern of the user. For example, as previously mentioned, the device can track one or more respiratory cycles during a certain period of time (e.g., via a respiration sensor and/or a cuff sensor). In the illustrated embodiment, the UI 1050 shows a graph of the tube pressure overlayed with a pair of horizontal lines, which can correspond to the upper threshold 812 and the lower threshold 814 illustrated in FIG. 8A. Accordingly, the UI 1050 can indicate to the user that the device is determining the upper threshold 812 and the lower threshold 814 while calibrating. In other embodiments, the UI 1050 can display other visuals to indicate other calculations performed by the device while calibrating (e.g., calculating an average pressure).

Referring next to FIG. 10F, the UI 1060 includes an indication that the device has been calibrated to the specific patient and the specific respiratory environment (e.g., the specific ventilator, ETT, tracheostomy tube, and/or the like) (“Calibrated”). The UI 1060 can also show both the minimum and maximum pressure thresholds (“20 cmH₂O” and “50 cmH₂O”), which may be input by the user or determined automatically by the device during calibration. Moreover, the UI 1060 can provide options for the user to edit either or both of the minimum and maximum pressure thresholds (“Edit”) or deflate the cuff immediately (“Deflate”). The bottom-left corner of the UI 1060 can also display the real-time cuff pressure (“30 cmH₂O”). Furthermore, the bottom-right corner of the UI 1060 can indicate which mode the device is currently in and allow the user to switch back to the static mode if desired.

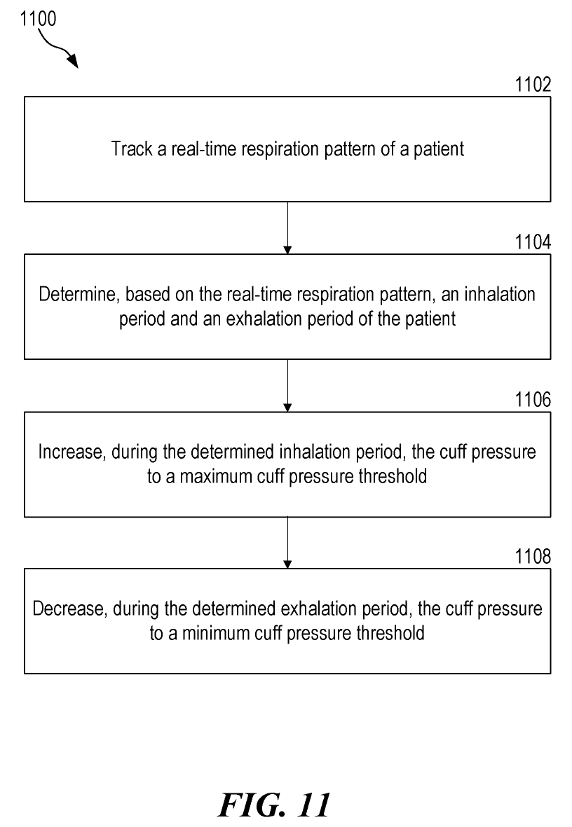

FIG. 11 is a flowchart illustrating a method 1100 for modulating cuff pressure in accordance with various embodiments of the present technology. While the operations of the method 1100 are described below in a particular order, one or more of the operations can be performed in a different order or omitted, and the method 1100 can include additional and/or alternative operations. Additionally, although the method 1100 may be described below with reference to the embodiments of the present technology described herein, the method 1100 can be performed with other embodiments of the present technology.

The method 1100 begins at block 1102 by tracking a real-time respiration pattern of a patient. For example, a respiration sensor can measure the pressure of airflow in an ETT. As another example, a cuff sensor can measure the cuff pressure. In either example, the sensor reading is expected to fluctuate in sync with the patient’s respiratory cycle.

At block 1104, the method 1100 continues by determining, based on the real-time respiration pattern, an inhalation period and an exhalation period of the patient. It is appreciated that the inhalation period and the exhalation period need not exactly match with the “true” inhalation and exhalation of the patient. Rather, the inhalation and exhalation periods can be determined according to the various methodologies described herein. Moreover, the inhalation and exhalation periods can be determined by (i) determining a real-time inhalation period and a real-time exhalation period and/or (ii) predicting a future inhalation period and a future exhalation period.

At block 1106, the method 1100 continues by increasing, during the determined inhalation period, the cuff pressure to a maximum cuff pressure threshold. For example, a cuff pump can be operated to pump fluid (e.g., air) into the cuff at a desired rate.

At block 1108, the method 1100 continues by decreasing, during the determined exhalation period, the cuff pressure to a minimum cuff pressure threshold. For example, a cuff pump can be operated to suction fluid (e.g., air) out of the cuff at a desired rate.

In some embodiments, the method 1100 also includes (i) entering, in response to a startup of the cuff pressure modulation device, a static mode of the cuff pressure modulation device, and (ii) maintaining, while the cuff pressure modulation device is in the static mode, the cuff pressure at a constant initial pressure (e.g., between 1–100 cmH₂O or between 20–30 cmH₂O, such as about 25 cmH₂O). In some embodiments, the method 1100 also includes (i) entering a calibration mode of the cuff pressure modulation device, and (ii) establishing, while the cuff pressure modulation device is in the calibration mode, baseline pressure readings that account for features specific to the patient, a ventilator that the ETT is coupled to, and an intubation environment.

In some embodiments, the method 1100 further includes receiving a user input specifying each of the minimum cuff pressure threshold and the maximum cuff pressure threshold. In other embodiments, the method 1100 further includes computing, based on the real-time respiration pattern of the patient, each of the minimum cuff pressure threshold and the maximum cuff pressure threshold.

In some embodiments, the method 1100 further includes (i) receiving an indication that the ETT is to be removed from the patient, and (ii) reducing the cuff pressure to a zero or near-zero pressure level. Thus, the cuff can be deflated for easy and safe removal from the trachea.

### IV. Examples

The present technology is illustrated, for example, according to various aspects described below as numbered examples (1, 2, 3, etc.) for convenience. These are provided as examples and do not limit the present technology. It is noted that any of the dependent examples may be combined in any combination, and placed into a respective independent example. The other examples can be presented in a similar manner.

1. A cuff pressure modulation device for modulating cuff pressure of a cuff integrated with an airway tube, the device comprising:

a pressure sensor configured to track a real-time respiration pattern of a patient;

a cuff pump configured to selectively inflate or deflate the cuff;

a memory storing a minimum cuff pressure threshold and a maximum cuff pressure threshold; and

a controller operably coupled to each of the pressure sensor, the cuff pump, and the memory, wherein the controller is configured to—

determine, based on the real-time respiration pattern, an inhalation period and an exhalation period of the patient,

operate, during the determined inhalation period, the cuff pump to increase the cuff pressure to the maximum cuff pressure threshold, and

operate, during the determined exhalation period, the cuff pump to decrease the cuff pressure to the minimum cuff pressure threshold.

2. The device of any of the examples herein, wherein the pressure sensor comprises a respiration sensor configured to measure pressure of airflow through the airway tube.

3. The device of any of the examples herein, wherein the pressure sensor comprises a cuff sensor configured to measure the cuff pressure in real-time.

4. The device of any of the examples herein, wherein the controller is further configured to determine, while the device is in a calibration mode, an average pressure reading from the pressure sensor, and wherein the controller is configured to determine the inhalation period and the exhalation period by comparing the real-time respiration pattern to the average pressure reading.

5. The device of any of the examples herein, wherein the controller is further configured to determine, while the device is in a calibration mode, an upper pressure threshold and a lower pressure threshold, and wherein the controller is configured to determine the inhalation period and the exhalation period by comparing the real-time respiration pattern to the average pressure reading and based on at least one of (i) rising and falling edge detection in an airway tube pressure to establish trending changes in breathing rate, (b) regression-based curve fitting to fit measured data to predetermined models of breathing patterns, accommodating both voluntary breathing and positive pressure-driven ventilation, (c) least-squares fitting as a curve-fitting approach to minimize the squared differences between model predictions and input sensor data, and/or (d) a machine learning approach to dynamically adjust a predictive model based on real-time input data, enabling patient-specific optimization.

6. The device of any of the examples herein, wherein the controller is further configured to determine, while the device is in a calibration mode, one or more baseline pressure thresholds based on at least two respiratory cycles of the real-time respiration pattern lasting no more than one minute.

7. The device of any of the examples herein, wherein the controller is configured to determine the inhalation period and the exhalation period by determining whether a pressure level of the real-time respiration pattern is increasing or decreasing.

8. The device of any of the examples herein, further comprising:

a cuff sensor configured to measure the cuff pressure in real-time;

a housing; and

a display screen disposed on one side of the housing, wherein the display screen is configured to display the cuff pressure measured by the cuff sensor in real-time.

9. The device of any of the examples herein, further comprising a cuff sensor configured to measure the cuff pressure in real-time, wherein the controller is further configured to:

detect that the cuff pressure has not reached the minimum cuff pressure threshold or the maximum cuff pressure threshold; and

alert a user in response to the detection.

10. The device of any of the examples herein, further comprising:

a discharge valve operably coupled to the cuff pump and configured to selectively control fluid flow from the cuff pump to the cuff; and

a suction valve operably coupled to the cuff pump and configured to selectively control fluid flow from the cuff to the cuff pump,

wherein the controller is operably coupled to each of the discharge valve and the suction valve and further configured to keep only one of the discharge valve or the suction valve open at a given moment.

11. The device of any of the examples herein, further comprising a pressure relief valve operably coupled to the cuff pump and configured to open in response to exceedance of a pressure threshold to avoid over-pressurizing the cuff.

12. The device of any of the examples herein, wherein the controller is configured to operate the cuff pump to increase the cuff pressure within 0.75 seconds from determining the inhalation period.

13. A method for modulating cuff pressure of a cuff integrated with an airway tube via a cuff pressure modulation device, the method comprising:

tracking a real-time respiration pattern of a patient;

determining, based on the real-time respiration pattern, an inhalation period and an exhalation period of the patient;

increasing, during the determined inhalation period, the cuff pressure to a maximum cuff pressure threshold; and

decreasing, during the determined exhalation period, the cuff pressure to a minimum cuff pressure threshold.

14. The method of any of the examples herein, further comprising:

entering, in response to a startup of the cuff pressure modulation device, a static mode of the cuff pressure modulation device;

prompting, while the cuff pressure modulation device is in the static mode, a user to input an initial cuff pressure between 0–100 cmH₂O; and

maintaining, while the cuff pressure modulation device is in the static mode, the cuff pressure at the initial cuff pressure.

15. The method of any of the examples herein, further comprising:

entering a calibration mode of the cuff pressure modulation device; and

establishing, while the cuff pressure modulation device is in the calibration mode, one or more baseline pressure readings that account for features specific to the patient and a respiratory assistance environment.

16. The method of any of the examples herein, wherein determining the inhalation period and the exhalation period comprises at least one of (i) determining a real-time inhalation period and a real-time exhalation period based on the real-time respiration pattern or (ii) predicting a future inhalation period and a future exhalation period based on the real-time respiration pattern.

17. The method of any of the examples herein, further comprising:

calibrating, while increasing or decreasing the cuff pressure, the cuff pressure modulation device based on the real-time respiration pattern of the patient;

updating, based on the calibration, the inhalation period and the exhalation period of the patient;

increasing, during the updated inhalation period, the cuff pressure to a second maximum cuff pressure threshold; and

decreasing, during the updated exhalation period, the cuff pressure to a second minimum cuff pressure threshold.

18. The method of any of the examples herein, further comprising receiving a user input specifying each of the minimum cuff pressure threshold and the maximum cuff pressure threshold.

19. The method of any of the examples herein, further comprising computing, based on the real-time respiration pattern of the patient, each of the minimum cuff pressure threshold and the maximum cuff pressure threshold.

20. The method of any of the examples herein, further comprising:

receiving an indication that the airway tube is to be removed from the patient; and

reducing the cuff pressure to a zero or near-zero pressure level.

### V. Conclusion

It will be apparent to those having skill in the art that changes may be made to the details of the above-described embodiments without departing from the underlying principles of the present disclosure. In some cases, well known structures and functions have not been shown or described in detail to avoid unnecessarily obscuring the description of the embodiments of the present technology. Although steps of methods may be presented herein in a particular order, alternative embodiments may perform the steps in a different order. Similarly, certain aspects of the present technology disclosed in the context of particular embodiments can be combined or eliminated in other embodiments. Furthermore, while advantages associated with certain embodiments of the present technology may have been disclosed in the context of those embodiments, other embodiments can also exhibit such advantages, and not all embodiments need necessarily exhibit such advantages or other advantages disclosed herein to fall within the scope of the technology. Accordingly, the disclosure and associated technology can encompass other embodiments not expressly shown or described herein, and the invention is not limited except as by the appended claims.

Where the context permits, singular or plural terms may also include the plural or singular term, respectively. For example, throughout this disclosure, the singular terms “a,” “an,” and “the” include plural referents unless the context clearly indicates otherwise. Moreover, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. Furthermore, as used herein, the phrase “and/or” as in “A and/or B” refers to A alone, B alone, and both A and B. Additionally, the terms “comprising,” “including,” “having,” and “with” are used throughout to mean including at least the recited feature(s) such that any greater number of the same features and/or additional types of other features are not precluded. Moreover, as used herein, the phrases “based on,” “depends on,” “as a result of,” and “in response to” shall not be construed as a reference to a closed set of conditions. For example, a step that is described as “based on condition A” may be based on both condition A and condition B without departing from the scope of the present disclosure. In other words, as used herein, the phrase “based on” shall be construed in the same manner as the phrase “based at least in part on” or the phrase “based at least partially on.”

As used herein, the use of relative terminology, such as “about”, “approximately”, “substantially” and the like refer to the stated value plus or minus ten percent. For example, the use of the term “about 90” refers to a range of from 110 to 110, inclusive. In instances in which the context requires otherwise and/or relative terminology is used in reference to something that does not include a numerical value, the terms are given their ordinary meaning to one skilled in the art. Additionally, all ranges disclosed herein are to be understood to encompass the endpoints, and any and all subranges subsumed therein. For example, a range of “1 to 9” includes any and all subranges between (and including) the minimum value of 1 and the maximum value of 9 (e.g., any and all subranges having a minimum value of equal to or greater than 1 and a maximum value of equal to or less than 9, such as 6.5 to 9).

Spatially relative terms (e.g., “beneath,” “below,” “over,” “under,” “above,” “upper,” “top,” “bottom,” “left,” “right,” “center,” “middle,” and the like) may be used herein for ease of description to describe one element’s or feature’s relationship relative to one or more other elements or features as illustrated in the figures. It will be understood that the spatially relative terms are intended to encompass different orientations of a device or system in use or operation, in addition to the orientation depicted in the figures. For example, if a device or system illustrated in the figures is rotated, turned, or flipped about a horizontal axis, elements or features described as “below” or “beneath” or “under” one or more other elements or features may then be oriented “above” the one or more other elements or features. Thus, the exemplary terms “below” and “under” are non-limiting and can encompass both an orientation of above and below. The device or system may additionally, or alternatively, be otherwise oriented (e.g., rotated ninety degrees about a vertical axis, or at other orientations) than illustrated in the figures, and the spatially relative descriptors used herein are interpreted accordingly. In addition, it will also be understood that when an element is referred to as being “between” two other elements, it can be the only element between the two other elements, or one or more intervening elements may also be present.

The disclosure set forth above is not to be interpreted as reflecting an intention that any claim or example requires more features than those expressly recited in that claim or example. Rather, as the preceding examples and the following claims reflect, inventive aspects lie in a combination of fewer than all features of any single foregoing disclosed embodiment. Thus, the preceding examples and the following claims are hereby expressly incorporated into the Detailed Description, with each claim standing on its own as a separate embodiment. This disclosure includes all permutations of the independent claims with their dependent claims.

What is claimed is:

1. A cuff pressure modulation device for modulating cuff pressure of a cuff integrated with an airway tube, the device comprising:
a pressure sensor configured to track a real-time respiration pattern of a patient;
a cuff pump configured to selectively inflate or deflate the cuff;
a memory storing a minimum cuff pressure threshold and a maximum cuff pressure threshold; and
a controller operably coupled to each of the pressure sensor, the cuff pump, and the memory, wherein the controller is configured to—
determine, based on the real-time respiration pattern, an inhalation period and an exhalation period of the patient,
operate, during the determined inhalation period, the cuff pump to increase the cuff pressure to the maximum cuff pressure threshold, and
operate, during the determined exhalation period, the cuff pump to decrease the cuff pressure to the minimum cuff pressure threshold.

2. The device of claim 1, wherein the pressure sensor comprises a respiration sensor configured to measure pressure of airflow through the airway tube.

3. The device of claim 1, wherein the pressure sensor comprises a cuff sensor configured to measure the cuff pressure in real-time.

4. The device of claim 1, wherein the controller is further configured to determine, while the device is in a calibration mode, an average pressure reading from the pressure sensor, and wherein the controller is configured to determine the inhalation period and the exhalation period by comparing the real-time respiration pattern to the average pressure reading.

5. The device of claim 1, wherein the controller is further configured to determine, while the device is in a calibration mode, an upper pressure threshold and a lower pressure threshold, and wherein the controller is configured to determine the inhalation period and the exhalation period by comparing the real-time respiration pattern to the average pressure reading and based on at least one of (i) rising and falling edge detection in an airway tube pressure to establish trending changes in breathing rate, (b) regression-based curve fitting to fit measured data to predetermined models of breathing patterns, accommodating both voluntary breathing and positive pressure-driven ventilation, (c) least-squares fitting as a curve-fitting approach to minimize the squared differences between model predictions and input sensor data, and/or (d) a machine learning approach to dynamically adjust a predictive model based on real-time input data, enabling patient-specific optimization.

6. The device of claim 1, wherein the controller is further configured to determine, while the device is in a calibration mode, one or more baseline pressure thresholds based on at least two respiratory cycles of the real-time respiration pattern lasting no more than one minute.

7. The device of claim 1, wherein the controller is configured to determine the inhalation period and the exhalation period by determining whether a pressure level of the real-time respiration pattern is increasing or decreasing.

8. The device of claim 1, further comprising:
a cuff sensor configured to measure the cuff pressure in real-time;
a housing; and
a display screen disposed on one side of the housing, wherein the display screen is configured to display the cuff pressure measured by the cuff sensor in real-time.

9. The device of claim 1, further comprising a cuff sensor configured to measure the cuff pressure in real-time, wherein the controller is further configured to:
detect that the cuff pressure has not reached the minimum cuff pressure threshold or the maximum cuff pressure threshold; and
alert a user in response to the detection.

10. The device of claim 1, further comprising:
a discharge valve operably coupled to the cuff pump and configured to selectively control fluid flow from the cuff pump to the cuff; and
a suction valve operably coupled to the cuff pump and configured to selectively control fluid flow from the cuff to the cuff pump,
wherein the controller is operably coupled to each of the discharge valve and the suction valve and further configured to keep only one of the discharge valve or the suction valve open at a given moment.

11. The device of claim 1, further comprising a pressure relief valve operably coupled to the cuff pump and configured to open in response to exceedance of a pressure threshold to avoid over-pressurizing the cuff.

12. The device of claim 1, wherein the controller is configured to operate the cuff pump to increase the cuff pressure within 0.75 seconds from determining the inhalation period.

13. A method for modulating cuff pressure of a cuff integrated with an airway tube via a cuff pressure modulation device, the method comprising:
tracking a real-time respiration pattern of a patient;
determining, based on the real-time respiration pattern, an inhalation period and an exhalation period of the patient;
increasing, during the determined inhalation period, the cuff pressure to a maximum cuff pressure threshold; and
decreasing, during the determined exhalation period, the cuff pressure to a minimum cuff pressure threshold.

14. The method of claim 13, further comprising:
entering, in response to a startup of the cuff pressure modulation device, a static mode of the cuff pressure modulation device;
prompting, while the cuff pressure modulation device is in the static mode, a user to input an initial cuff pressure between 0–100 cmH₂O; and
maintaining, while the cuff pressure modulation device is in the static mode, the cuff pressure at the initial cuff pressure.

15. The method of claim 13, further comprising:
entering a calibration mode of the cuff pressure modulation device; and
establishing, while the cuff pressure modulation device is in the calibration mode, one or more baseline pressure readings that account for features specific to the patient and a respiratory assistance environment.

16. The method of claim 13, wherein determining the inhalation period and the exhalation period comprises at least one of (i) determining a real-time inhalation period and a real-time exhalation period based on the real-time respiration pattern or (ii) predicting a future inhalation period and a future exhalation period based on the real-time respiration pattern.

17. The method of claim 13, further comprising:
calibrating, while increasing or decreasing the cuff pressure, the cuff pressure modulation device based on the real-time respiration pattern of the patient;
updating, based on the calibration, the inhalation period and the exhalation period of the patient;
increasing, during the updated inhalation period, the cuff pressure to a second maximum cuff pressure threshold; and
decreasing, during the updated exhalation period, the cuff pressure to a second minimum cuff pressure threshold.

18. The method of claim 13, further comprising receiving a user input specifying each of the minimum cuff pressure threshold and the maximum cuff pressure threshold.

19. The method of claim 13, further comprising computing, based on the real-time respiration pattern of the patient, each of the minimum cuff pressure threshold and the maximum cuff pressure threshold.

20. The method of claim 13, further comprising:
receiving an indication that the airway tube is to be removed from the patient; and
reducing the cuff pressure to a zero or near-zero pressure level.
